(19) United States
(12) Patent Application Publication
(10) Pub. No.: US 2017/0224667 A1
(43) Pub. Date: Aug. 10, 2017

(54) CANCER CHEMOPREVENTIVE AGENT

(71) Applicants: Arata AZUMA, Mitaka-shi, Tokyo (JP); KDL, Inc., Chiyoda-ku, Tokyo (JP)

(72) Inventors: Arata AZUMA, Tokyo (JP); Yukiko MIURA, Tokyo (JP)

(21) Appl. No.: 15/519,360

(22) PCT Filed: Apr. 21, 2015
(86) PCT No.: PCT/JP2015/062117

§ 371 Date: Apr. 14, 2017

(30) Foreign Application Priority Data
Oct. 15, 2014 (JP) 2014-210690

(51) Int. Cl. A61K 31/4418; A61K 45/06

(52) U.S. Cl. CPC A61K 31/4418; A61K 45/06

(57) ABSTRACT

Provided is a medicine for cancer chemoprevention, the medicine being characterized by containing, as an active ingredient, pirfenidone or a pharmaceutically acceptable salt thereof.

### TECHNICAL FIELD

This invention is in the field of cancer chemoprevention. More precisely, this invention is to provide the method of cancer chemoprevention using the drug containing pirfenidone as a pharmacological active ingredient. There are also provided the use of pirfenidone for the production of a medicament containing pirfenidone for cancer prevention, and methods for cancer chemoprevention comprising administering an effective amount of pirfenidone for cancer prevention to mammals including human.

### BACKGROUND ART

Cancer is the number one cause of death in our country and a disease which occupies a high rank as cause of death in the developed world.

For the treatment of cancer, there are surgery, radiotherapy, chemotherapy and immune therapy, and these are appropriately combined and then applied. Rapid progress has been made in cancer treatments, especially in drug therapy. Molecular target drugs are currently available in addition to chemotherapeutic drugs and they have dramatically improved the outcome in some cancers. Drug therapy is the sole treatment method for patients who developed systemic metastasis, but there is a limitation in their effectiveness. Therefore, new drug is needed.

Recently cytostatic anticancer agents such as anti-agiogenesis, which inhibit cancer growth indirectly by inhibiting angiogenesis as well as cytotoxic agents which inhibit cancer cell growth directly. Supporting drugs such as antiemetic drugs and G-CSF, which reduce side effects caused by chemotherapeutic agents have been available.

Furthermore, cancer painkillers improve the quality of life (QOL) of the patient and, as a result, help improvement of the cancer treatment.

On the other hand, trials to prevent cancer have been widely investigated. The 12 cancer prevention method including smoking cessation, the intake of lots of vegetables, moderate drinking, and lifestyle improvements including a limit of salt intake has been issued by the Japanese Cancer Association and the activity of cancer prevention is enlightened. In addition, aggressive intervention with drugs for preventing cancer, so-called “cancer chemoprevention” are sought and investigated. This is accomplished not only with animal experiments but also in clinical studies. Inspection of toxicity and effectiveness in cancer prevention and decisions of optimum doses are necessary for cancer prevention studies. It is very difficult to develop a chemopreventive agent because of the evaluation difficulty including decrease in incidence rate in humans as well as in animal models and the optimal dose finding with less toxicity.

Several clinical trials of the compounds, mainly natural compounds, which were found to have their chemopreventive effects in animal models, were conducted for subjects who have high risk factor of carcinogenesis such as smoking, colon polyp and so forth based on systemic epidemiological studies in humans. The only compounds with confirmed chemopreventive effects are tamoxifen and raloxifen in prevention in breast cancer (non-patent reference 1). A chemopreventive effect of β-carotene on lung cancer was reported in animal studies, but no effects on lung cancer were found in smokers and no-smokers, and it rather lead to an increase in lung cancer risk (non-patent document 2-3). Although a certain number of new compounds have been reported to show chemopreventive activities, none of them has shown effects in human. (patent document 1-6).

Pirfenidone, 5-methyl-1-phenyl-2-(1H)-pyridone, is widely known as an effective drug for the prevention and treatment for diseases relevant to fibrosis (patent document 7), and especially useful for the treatment such as fibrosis lesions, transmissibility warts, contact-related dermatitis, a keloid, pulmonary fibrosis, the fibrosis enlargement of the prostate, restoration and prevention of nephrosclerosis and so forth, or post-surgery burn injury, and Alzheimer's disease. Pirfenidone was first approved as pirespa® 200 mg tablet for the treatment of idiopathic pulmonary fibrosis in Japan in 2008 by the present inventors. In addition, it was approved for the treatment of idiopathic pulmonary fibrosis in Europe in 2010, and NDA was filed in the United States in 2014.

Pirfenidone is expected to inhibit cancer growth, because pirfenidone inhibits beta type variation growth factor (transforming growth factor-β) and epithelium mesenchyma transition (EMT). Burghadtt et al. reported that pirfenidone inhibited proliferation of the malignant glioma cells (non-patent document 4). Kozono reported the inhibitory effects on fibrosis caused by pancreatic cancer (non-patent document 5).

Idiopathic pulmonary fibrosis is often associated with lung cancer and such patients can be considered to be at high risk of developing lung cancer. Surgery of lung cancer of the patients with idiopathic pulmonary fibrosis is known to cause a development of a serious acute exacerbation. There is a report that this acute exacerbation was inhibited by pre-treatment with pirfenidone (non-patent document 6).

### CITATION LIST

### Patent Literatures

[Patent literature 1] JP2014-55196A

[Patent literature 2] JP2014-50390A

[Patent literature 3] JP2010-138192A

[Patent literature 4] JP2012-510965A

[Patent literature 5] JP2006-510658A

[Patent literature 6] JP2002-510667A

[Patent literature 7] JPH02-215719A

### Non-Patent Literatures

[Non-patent literature 1] Advani P, Moreno-Aspitia A. Current strategies for the prevention of breast cancer. Breast Cancer (Dove Med Press). 2014; 6: 59-71.

[Non-patent literature 2] IARC. IARC Handbook of Cancer Prevention Volume 2 “Cartenoids”, IARC Press, Lyon (1989).

[Non-patent literature 3] Greenberg A. K. , Tsay J. C., Tchou-Wong K. M., Jorgensen A, Rom W. N. Chemoprevention of lung cancer: prospects and disappointments in human clinical trials. Cancers (Basel). 2013; 5: 131-48.

[Non-patent literature 4] Burghardt I, Tritschler F, Opitz C A, Frank B, Weller M, Wick W. Pirfenidone inhibits TGF-beta expression in malignant glioma cells. Biochem Biophys Res Commun. 2007; 354: 542-7.

[Non-patent literature 5] Kozono S, Ohuchida K, Eguchi D, Ikenaga N, Fujiwara K, Cui L, Mizumoto K, Tanaka M. Pirfenidone inhibits pancreatic cancer desmoplasia by regulating stellate cells. Cancer Res. 2013; 73: 2345-56.

[Non-patent literature 6] Successful Resection of Lung cancer with idiopathic pulmonary fibrosis treated by pirfenidone during perioperative period. Jap J. Thorac Surg. 2013; 66, 890-893.

### SUMMARY OF INVENTION

### Technical Problem

The aim this invention is to provide a medicament of cancer chemoprevention.

### Solution to Problem

The present inventors had hypothesized that pirfenidone might have a cancer chemoprevention activity based on the results that show that pirfenidone can normalize damaged tissue such as inhibitory effects on scars, in addition to its antifibrotic action. Thus, the present inventors evaluated the cancer chemoprevention effect of pirfenidone in idiopathic pulmonary fibrosis patients who are said to have a high risk factor of carcinogenesis. The patients were randomly assigned to a pirfenidone group and a non-pirfenidone group, and the pirfenidone group was observed for more than 12 months. As a result, the present inventors have found that pirfenidone has a cancer chemoprevention activity and accomplished the present invention. This efficacy was not explained by its effectiveness on idiopathic pulmonary fibrosis, and is therefore a novel activity of pirfenidone.

To describe more specifically, the present invention is as follows:

(1) A medicament of cancer chemoprevention which comprises pirfenidone or pharmacologically acceptable salt thereof as a pharmaceutical active ingredient.

(2) A medicament of cancer chemoprevention which comprises 5-methyl-1-phenyl-2-(1H)-piridone as a pharmaceutical active ingredient.

(3) The medicament of cancer chemoprevention according to (1) or (2) wherein said medicament is for cancer chemoprevention of solid cancer.

(4) The medicament of cancer chemoprevention according to (3) wherein said solid cancer is selected from the group consisting of a brain tumor, spinal cord tumor, oral cancer, pharyngeal cancer, cancer of nose, cancer of larynx, thyroid cancer, lung cancer, breast cancer, a mediastinum tumor, a mesothelioma, cancer of esophagus, stomach cancer, duodenal small intestine cancer, colon cancer, GIST, liver cancer, cholangiocarcinoma, a gallbladder cancer, pancreatic cancer, kidney cancer, urinary tract cancer, bladder cancer, adrenal tumor, prostate cancer, testicular cancer, cervical cancer, endometrial cancer and ovarian cancer.

(5) The medicament of cancer chemoprevention according to (3) or (4) wherein said solid cancer is lung cancer.

(6) The medicament of cancer chemoprevention according to any one of (1) to (5) wherein said medicament is for cancer chemoprevention for a subject who has a high risk factor of carcinogenesis.

(7) The medicament of cancer chemoprevention according to (6) wherein said high risk factor of carcinogenesis is idiopathic pulmonary fibrosis.

(8) The medicament of cancer chemoprevention according to any one of (1) to (7) further comprising any other pharmaceutical active drug as a concomitant drug.

(9) The medicament of cancer chemoprevention according to (8) wherein said any other pharmaceutical active drug is a drug which prevents carcinogenesis, inhibits indirectly growth of cancer and/or directly inhibits growth of cancer.

(10) The medicament of cancer chemoprevention method of cancer chemoprevention according to (8) wherein said any other pharmaceutical active drug is used as adjuvant chemotherapeutic drug.

(11) An oral or parenteral pharmaceutical composition comprising an effective amount of pirfenidone together with one or more pharmaceutically acceptable additive(s).

(12) A method of cancer chemoprevention which comprises a step of administering an effective amount of pirfenidone for cancer prevention to mammals including human.

(13) A method of cancer chemoprevention which comprises a step of administering an effective amount of pirfenidone for cancer prevention to a subject who has a high risk factor of carcinogenesis.

(14) The method of cancer chemoprevention according to (13), wherein said high risk factor of carcinogenesis is idiopathic pulmonary fibrosis.

(15) The method of cancer chemoprevention according to any one of (12) to (14), wherein any other pharmaceutical active drug is further administered as a concomitant drug.

(16) The method of cancer chemoprevention according to (15), wherein said any other pharmaceutical active drug is a drug which prevents carcinogenesis, inhibits indirectly growth of cancer and/or directly inhibits growth of cancer.

(17) The method of cancer chemoprevention according to (15), wherein said any other pharmaceutical active drug is used as adjuvant chemotherapeutic drug.

### Advantageous Effects of Invention

According to the favorable findings of the present invention as mentioned above, it provides a cancer chemoprevention of solid carcinoma such as a brain tumor, spinal cord tumor, oral cancer, pharyngeal cancer, cancer of nose, cancer of larynx, thyroid cancer, lung cancer, breast cancer, a mediastinum tumor, a mesothelioma, cancer of esophagus, stomach cancer, duodenal small intestine cancer, colon cancer, GIST, liver cancer, cholangiocarcinoma, a gallbladder cancer, pancreatic cancer, kidney cancer, urinary tract cancer (renal pelvis cancer, ureter cancer), bladder cancer, adrenal tumor, prostate cancer, testicular cancer, cervical cancer, endometrial cancer and ovarian cancer.

In other embodiment of the present invention, there are also provided the use of pirfenidone for the production of a medicament containing pirfenidone for cancer prevention, and methods for cancer chemoprevention comprising administering an effective amount of pirfenidone for cancer prevention to mammals including human.

The medicament of the present invention provides prevention of cancerogenesis in healthy human with a high risk of cancer, and patients with idiopathic pulmonary fibrosis, idiopathic interstitial pneumonia, COPD, Helicobacter pylori-positive gastritis, hepatitis C virus-positive hepatitis and cirrhosis.

### BRIEF DESCRIPTION OF THE DRAWINGS

FIG. 1 shows time course of unaffected rate of lung cancer in patients with idiopathic pulmonary fibrosis treated with/without pirfenidone.

### DETAILED DESCRIPTION OF THE PREFERRED EMBODIMENTS

The active ingredient contained in the medicament of this invention is 5-methyl-1-phenyl-2-(1H)-pyridone (pirfenidone) represented by the structure (1) or its pharmaceutically acceptable salt thereof.

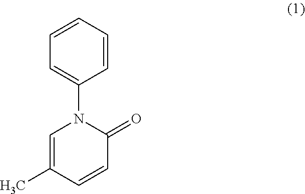

The pharmaceutically acceptable salts include salt(s) with acid and alkaline. As an acid forming the salt with pirfenidone, there are hydrochloric acid, sulfuric acid, phosphoric acid, p-toluenesulfonic acid, and methanesulfonic acid. As the alkaline salt of pirfenidone, there are sodium salt and potassium salt.

The medicament of the present invention can be used for cancer chemoprevention. The term “cancer chemoprevention” means preventing the development of cancer by drugs. The present invention is a class of “cancer chemopreventive drug”. “Effective dose of cancer chemoprevention” in the present invention means the dose for presenting the preventive effect on the targeted disease, i.e. cancer preventive or preventing the development of malignancy, or the dose detecting the cancer chemopreventive effect.

The medicament of the present invention is used for cancer chemoprevention for human with risk factors of carcinogenesis. More preferably, although not particularly limited, the medicament of this invention is used for healthy human with a high risk of cancer. This invention is also used for the patients who suffer from a cancer in order to enhance anti-cancer activity with combination of cancer treatment drugs.

The “subjects with risk factors of carcinogenesis” means healthy human with a high risk of cancer, and patients with idiopathic pulmonary fibrosis, idiopathic interstitial pneumonia, COPD Helicobacter pylori-positive gastritis, hepatitis C virus-positive hepatitis and cirrhosis.

There is no need to limit the high risk factors of cancers, but there are provided the factors including smoking (actively), smoking (passively), hyperplasia and metaplasia of organs such as the pulmonary bronchus, liver, fibrosis such as idiopathic pulmonary fibrosis, air pollution, occupational revelation such as chrome, arsenic, nickel, or asbestos, drinking alcohol, being overweight and obesity, lack of exercise, lack of vegetables and lack of fruit, salt intake, Helicobacter pylori infection, hepatitis C (HCV) and hepatitis B (HBV) infection, humans papilloma virus (HPV) infection, type I human T cell leukemia virus (HTLV-I) infection, Epstein-Barr virus (EBV) infection, exogenous hormone use, hormone replacement therapy (HRT), oral contraceptive (OC). Smoking (actively) is a risk factor for oral cavity and pharyngeal cancer, esophagus cancer, stomach cancer, colorectal cancer, liver cancer, pancreatic cancer, larynx cancer, lung cancer, cervical cancer, ovarian cancer, bladder cancer, renal cancer, and myeloid leukemia; smoking (passively) and pulmonary fibrosis are risk factors for lung cancer (non-smoker); drinking alcohol is a risk factor for oral cavity and pharyngeal cancer, esophagus cancer, colorectal cancer, liver cancer, and female breast cancer; being overweight and obesity are risk factors for colorectal cancer, pancreatic cancer, postmenopausal breast cancer, endometrial cancer, and kidney cancer; lack of exercise is a risk factor for colorectal cancer, breast cancer, and endometrial cancer; lack of vegetables and lack of fruit are risk factors for esophagus cancer, stomach cancer, and lung cancer; salt intake is a risk factor for gastric cancer; Helicobacter pylori infection is a risk factor for stomach (non-cardiac part) cancer; hepatitis C virus (HCV) and hepatitis B virus (HBV) infections are a risk factor for liver cancer; human papilloma virus (HPV) infection is a risk factor for oral cavity cancer, oropharyngeal cancer, anal cancer, penile cancer, vaginal cancer, vulval cancer, and cervical cancer; type I human T cell leukemia virus (HTLV-I) infection is a risk factor for adult T cell lymphoma/leukemia (ATL); Epstein-Barr virus (EBV) infection is a risk factor for nasopharyngeal cancer Burkitt lymphoma, Hodgkin lymphoma; and use of exogenous hormones, hormone replacement therapy, and oral contraceptive (OC) are risk factors for breast cancer in women.

Recently, a genetic test is also used as a method to know the cancer-causing risk. There is no need to limit, but onset genes of cancer include AFP, BAGE, BCL2, CA-125, CALCA, CD44, CEA, CGA, c-Kit, c-met, c-myc, COX2, CyclinD1, Cytokeratin-19, Cytokeratin-20, Cytokeratin-7, E2F1, E2F3, EEF1A2, EGFR, EVI-1, FGFR2(K-sam), GAGE, Gli1GPC3, HBV, HCCR, HCGbeta, HCV, Her-2/neu, HIF1α, HnRNPA2/B1, HPV, hTERT, HTLV, L-myc, MAGE-A1, MAGE-A12, MAGE-A3, A6, MAGE-A4, MDM2, MDR-1, MMP-2, MMP-9, Mucin1, Mucin4, Mucin7, NCOA4, N-myc. NSE, ProGRP, PSA, PSMA, RCAS1, SCC, survivin, Thyroglobulin, VEGF-A, VEGF-C and WT-1. Mutated genes include P53, K-RAS, H-RAS, N-RAS, BRAF, APC, and EGF receptor gene, and Methylation genes includes APC, ATM, BRCA1, DCC, E-Cadherin, H-cadherin, hMLH1, p14, p15, p16, RAR-Beta2, RASSF1A, RB1, TIMP3.VH. These genes are used for genetic screenings. Patients can be identified as treatment candidates for the medicament of the present invention based on these genetic screenings.

The medicament of the present invention can be used for treatment of patients suffering from cancer by using pirfenidone or its pharmaceutically acceptable salt alone or combination with other pharmaceutical active drugs in order to prevent malignant alteration of cancer or to enhance suppression of cancer growth.

There is no need to limit the pharmaceutically active ingredients for combinations with pirfenidone, but more preferably there are provided compounds and drugs which prevent carcinogenesis, inhibit indirectly growth of cancer, and/or directly inhibit growth of cancer. Especially, combination of pirfenidone with chemotherapeutic drugs which directly inhibit growth of cancer is used for preventing recurrence of cancer or secondary cancer. Combination of pirfenidone with chemotherapeutic drugs are more specifically useful as an adjuvant therapy.

As for the chemopreventive drug, there are no clinical available drugs, but there are provided the compounds which are known to show chemopreventive activities in animal models. Those includes , not particularly limited, but more preferably are carotenoids such as β-carotene, α-carotene, lycopenes, luteins, fucoxanthins; retinoids such as retinoic acids, 13-cis-retinoic acid, 9-cis-retinoic acid, fenretinide, ethylate, acitretin, N-ethyl-retinamide, and targretin ; anti-oxidants such as tocopherol, vitamin E, selenium and N-acetyl cystein; green tea components such as catechins; and NSAIDs such as aspirin, sulindac, piroxicam, and indometacin.

As for the compounds indirectly inhibit growth of cancer, there is no need to limit, but more preferably are provided anti-agenesis drugs such as bevacizumab (Avastin) and thalidomide (Thaled).

There is no need to limit, but are provided chemotherapeutic drugs and molecular targeted drug as cancer treatment drug which directly inhibit growth of cancer. As for the chemotherapeutic drugs, there are provided alkylating agents such as ifosfamide (Ifomide), cyclophosphamide (Endoxane), dacarbazine (Dacarbazine), temozolomide (Temodar), nimustine (Nidolan), busulfan (Busulfex, Mablin), procarbazine (procarbazine hydrochloride), melphalan (Alkeran), and ranimustine (Cymerin); anti metabolites such as enocitabine (Sanrabin), capecitabine (Xeloda), carmofur (Mifrol), cladribine (Leustatin), gemcitabine (Gemzar), cytarabine (Cylocide), cytarabine ocfosphate (Starasid), tegafur (Atyron, Aftoful, Tefseal, Futrafur, Lunasin), combination of tegafur and uracil (UFT), combination of tegafur, gimeracil and oteracil potassium (TS-1), doxifluridine (Furtulon), nelarabine (Aranon G), hydroxycarbamide (Hydrea), fluorouracil (5-FU, Calzonar, Bennan, Lunachol, Lunapon), fludarabine (Fludara), pemetrexed (Alimta), pentostatin (Coforin), mercaptopurine (Leukerin), methotrexate (Methotrexate), and trifluridine.tipiracil hydrochloride (Lonsurf);

anticancer antibiotics such as actinomycin D (Cosmegen), aclarubicin (Aclacinon), Amrubicin (Calsed), idarubicin (Idamycin), epirubicin (epirubicin hydrochloride, Farmorubicin), zinostatinstimalamer (Smancs), daunorubicin(Daunopmycin), doxorubicin (Adriacin), pirarubicin (Pinorubin, Terarubicin), bleomycin (Bleo), peplomycin (Pepleo), mitomycin C (Mitomycin), mitoxantrone (Novantron), and liposomaldoxorubicin (Doxil); plant alkaloids such as irinotecan (Campto, Topotecin), etoposide (VePesid, Lastet), eribulin (Halaven), sobuzoxane (Perazolin), docetaxel (Taxotere), nogitecan (Hycamtin), paclitaxel (Taxol), paclitaxel injection (Abraxane), Vinorelbine (Navelbine), vincristine (Oncovin), vindesine (Fildesin), and vinblastine (Exal); platinum compounds such as oxaliplatin (Elplat), carboplatin (Carboplatin, Carbomerck, Paraplatin), cisplatin (IA-call, Conabri, Cisplatin, etc.), and nedaplatin (Aqupla); hormonal agents such as anastrozole (Arimidex), exemestane (Aromasin), estramustine (Estracyt, Biasetyl, Proesta), ethinylestradiol (Prosexol), chlormadinone (Aptacor, Papacor, Prostal, Prostat, etc.), goserelin (Zoladex), tamoxifen (Advan, Emarc, Nolvadex, etc.), dexamethasone (Orgadrone, Dexamethosasone Elixir, Methaderm), toremifene (Toremifane, Fareston), bicalutamide (Casodex), flutamide (Odyne, Flutamide, Flutamerck), prednisolone (prednisolone, Predonine, Predovan, etc.), fosfestrol (Honvan), mitotane (Opeprim), methyltestosterone (Enarmon, Enerfa), medroxyprogesterone (HysronH, Progeston) , mepitiostane (Thioderon), leuprorelin (Leuplin), and letrozole (Femara); and biological response modifiers such as interferon α (IFN α, OIF, Sumiferon, etc.), interferon β (IFN-Mochida, Feron), interferon γ (Imunomax γ, OH-γ, Biogamma), interleukin (Imunace, Celeuk), ubenimex (Bestatin), dried BCG vaccine (Immunobladder, ImmuCyst), and lentinan (Lenakat, Lentinan). As for the morecular target drugs, there are provided ibritumomabtiuxetan (Zevalin), imatinib (Glivec), everolimus (Afinitor), erlotinib (Tarceva), gefitinib (Iressa), gemtuzumab ozogamicin (Mylotarg), sunitinib (Sutent), cetuximab (Erbitux), sorafenib (Nexavar), dasatinib (Sprycel), tamibarotene (Amnolake), trastuzumab (Herceptin), tretinoin (Vesanoid), panitumumab (Vectibix), bortezomib (Velcade), lapatinib (Tykerb), and rituximab (Rituxan).

As for the adjuvant therapeutic drugs, there are provided UFT (tegafur-uracil)+leucovorin, wherein “+” means combination, capecitabine (Xeloda), FOLFOX therapy (5-fluorouracil+leucovorin+oxaliplatin), XELOX therapy (capecitabine+oxaliplatin) for colorectal cancer; cisplatin or carboplatin+vinorelbine or paclitaxel, and UFT (tegafur-uracil) for non small cell lung cancer; TS-1 (tegafur-gimeracil-oteracil potassium), oral fruoropyrimidines such as UFT(egafur-uracil), mytomycin C+oral fruoropyrimidines, and 5-fluorouracil+leucovori for gastric cancer; CMF therapy (cyclophosphamide+methotrexate+5-fluorouracil), AC therapy (anthracyclines such as doxorubicin and epirubicin+cyclophosphamide), taxans such a paclitaxel and docetaxel, oral furuoropyrimidines such as UFT, anti-estrogens such as tamoxifen and tolemifen, aromatase inhibitors such as anastrozole, exemestane, and letrozole, goserelin, luteinizing hormone-releasing hormones (LH-RH) compounds such as leuprorelin acetate, anti-HER2 monoclonal antibodies such as trastuzumab and pertuzumab, anti-VEGF monoclonal antibodies such as bevacizumab, and low molecular target compounds such as everolimus, lapatinib, and sorafenib for breast cancer; interferon α for liver cancer; cisplatin+5-fluorouracil for esophageal cancer; gemcitabine, TS-1 (tegafur-gimeracil-oteracil potassium), and capecitabine for biliary tract cancer; radiation+5-fluorouracil, 5-fluorouracil+leucovori, 5-fluorouracil+doxorubicin+mytomycin C, and gemcitabine for pancreatic cancer; TC therapy (taxans+cyclophosphamide) for ovarian cancer; radiation for uterine cancer; and paclitaxel+gemcitabine+cisplatin for bladder cancer.

The medicament of the present invention can be administered as pirfenidone alone, but preferably administered as oral or non-oral pharmaceutical composition, which are prepared by the known methods.

As for the appropriate pharmaceutical composition for oral administration, for example there are provided tablets, capsules, fine granules, granules, liquid and solutions, and syrups. As the appropriate pharmaceutical composition for non-oral administration, for example there are provided injections, suppositories, inhalations, ophthalmic solutions, nasal drops, ointments, creams, patches and so forth. As for the preferable dosage form of the medicament of this invention, there are pharmaceutical compositions for oral administration.

The pharmaceutical composition of this invention is prepared by adding one or more pharmacologically or pharmaceutically acceptable additive(s) such as a diluting agent, a disintegrating or collapsing agent, a binder, a lubricant, a coating agent, a pigment, a diluent, a basis, a dissolving or a solubilizing agent, a tonicity adjusting agent, a pH modifier, a stabilizer, a aerosolized agent and an adhesive, but they are not limited to these.

As for the preparation of the pharmaceutical composition for oral administration including tablets, capsules, granules, and fine granules, diluting agents such as lactose, crystalline cellulose, and starch; lubricants such as magnesium stearate and talc; binders such as hydroxypropyl cellulose and polyvinylpyrrolidone; disintegrator such as carboxymethylcellulose calcium, low substituted hydroxypropylmethylcellulose; and coating agents such as hydroxypropylmethylcellulose, macrogol, and silicone resins can be used as needed. As for the preparation of the pharmaceutical composition for ophthalmic solutions, tonicity adjusting agents such as sodium chloride, potassium chloride and concentrated glycerin; buffering agents such as sodium phosphate, sodium acetate, boric acid, and monoethanol amine; stabilizers such as sodium citrate and disodium edetate; preservatives such as benzalkonium chloride and p-hydroxybenzoic ester; surfactants such as polysorbate 80, and polyoxyethylene hydrogenated castor oil; and pH modifiers such as diluted hydrochloric acid and sodium hydroxide can be used as needed. The pH of the ophthalmic solutions of the invention, not particularly limited, but is more preferably in the range between 4 and 8, which is permitted as an ophthalmic solution.

Effective dosage of the medicament of this invention, not specifically limited, and is decided according to conditions such as patient conditions, age, and body weight, route of administration, and kinds of active ingredient drug. In one embodiment, when pirfenidone is administered orally, the daily dose is in the range from 50 mg to 3000 mg, preferably 10 mg to 1000 mg, and is administered one time to several times a day. The above described dosage is for exemplification and can be adjusted arbitrarily.

The dosage regimen is adjusted when the medicament of the present invention is used for cancer chemoprevention alone or with combination of other treatment drugs for chemoprevention. Specifically, the optimal dosage regimen of pirfenidone is determined based on each of the combined treatment drugs. The dose of pirfenidone is preferably lowest dose which is more effective and less toxic, since the medicament of the present invention is used for prevention, and hence duration of the treatment is long.

### EXAMPLES

The following examples are set forth so as to provide a complete disclosure and description of how to make and use the present invention. The scope of the present invention is not limited to the following examples.

A total of 319 idiopathic pulmonary fibrosis patients that did not develop lung cancer were randomly allocated into two groups. 87 in the pirfenidone group (83 idiopathic pulmonary fibrosis, four nonspecific interstitial pneumonia) and 232 in the non-pirfenidone group (213 idiopathic pulmonary fibrosis, 18 nonspecific interstitial pneumonia, and one patient of desquamative interstitial pneumonia). The patients in the pirfenidone group received pirfenidone for 12-75 months (an average of 27.9±14.7 months). As a result, the incidence of lung cancer in the pirfenidone group was 2, whereas in the non-pirfenidone group it was 49. The duration of observation period in the pirfenidone group was 65.5±42.6 months, whereas in the control group it was 57.1±37.5 months. In addition, among 73 deaths in the non-pirfenidone group, 11 (4.7%) patients died by lung cancer, whereas among 29 deaths in the pirifenidone group, one (1.2%) patient died by lung cancer. These data indicate the chemopreventive effect of pirfenidone on lung cancer.

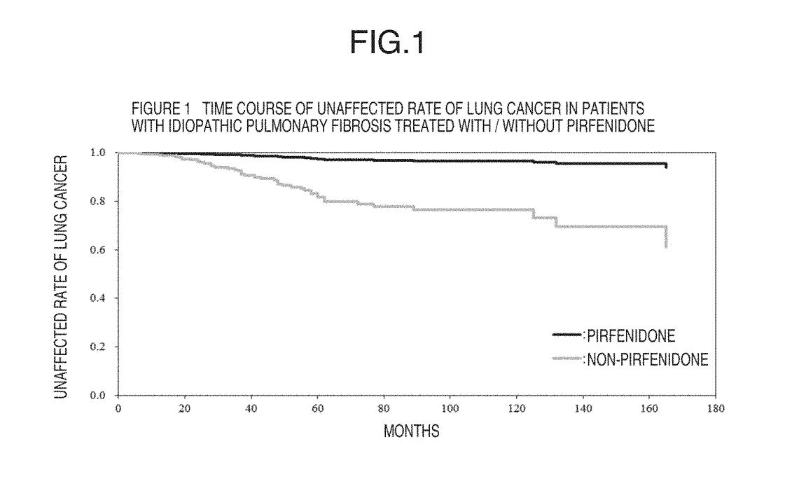

Results are shown in Table 1 and FIG. 1.

TABLE 1
Result of lung cancer prevention through pirfenidone medication

| | Pirfenidone | Non-pirfenidone | |
|---|---|---|---|
| | group | group | p-value^{#} |
| Number of patients | 87 | 232 | NS |
| Observation period | 65.5 ± 42.6 | 57.1 ± 37.5 | NS |
| (months) | (range: 13-215) | (range: 12-252) | |
| | months | months | |
| Treatment (months) | 27.9 ± 14.7 | — | — |
| | (range: 12-75) | | |
| | months | | |
| Incidence of lung | 2 (2.3%) | 49 (21.1%) | <0.000l* |
| cancer, N (%) | | | |
| Deaths, N (%) | 29 (33.3%) | 73 (31.5%) | NS |
| Death by cancer, N (%) | 1 (1.1%) | 11 (4.7%) | NS |

^{#}Chi-squared test
*p < 0.05

FIG. 1 shows time course of incidence of lung cancer in patients with idiopathic pulmonary fibrosis treated with/without pirfenidone. The accumulated incidences of lung cancer in patients with idiopathic pulmonary fibrosis not treated with pirfenidone after 1, 5, and 10 years were 0.9%, 18.3%, and 26.8%, whereas in patients treated with pirfenidone after 1, 5, and 10 years the accumulated incidences of lung cancer were 0%, 2.5%, and 6.4%.

The risk factors of incidence of lung cancer were analyzed using logistic regression analysis. As for the variables, backgrounds such as age, sex, number of packs of cigarettes smoked per day by the number of years the person has smoked (pack year), current smoking, pulmonary emphysema, stage of idiopathic pulmonary fibrosis, lung functions (% vital capacity(VC), and forced expiratory volume in 1 second as percent of forced vital capacity; FEV 1%), respiratory function (carbon monoxide lung diffusing capacity; % DLCO), and arterial oxygen partial pressure (PAO₂); home oxygen therapy (use/no use), pirfenidone (use/no use), prednisolone (use/no use), N-acetyl cysteine (use/no use) and so forth were used. As a result, pack year, current smoking and pulmonary emphysema were found to be factors which significantly increase the risk factors of incidence of lung cancer, while pirfenidone, N-acetyl cysteine and lung function (FEV 1%) were found to be factors which significantly decrease the risk factors of incidence of lung cancer. Then, the factors affecting the incidence of lung cancer were analyzed using multivariate Cox proportion hazard mode. The results are presented in Table 2. Four variables were found as affecting factors. Among them, current smoking and pack year were the factors increasing the risk of lung cancer, whereas treatment with pirfenidone was only factor decreasing the risk of lung cancer. This supports the lung cancer protective efficacy of pirfenidone.

TABLE 2
Risk of lung cancer in patients with idiopathic pulmonary fibrosis#

| | | 95% | | |
|---|---|---|---|---|
| | | confidence interval | | |
| Variable | Hazard ratio | LCL | UCL | p-value |
| Pirfenidone | 0.128 | 0.031 | 0.535 | 0.0048* |
| Current smoking | 1.122 | 1.037 | 1.213 | 0.0041* |
| Pack years | 2.629 | 1.457 | 4.743 | 0.0013* |
| % of Pulmonary | 1.021 | 1.003 | 1.038 | 0.0219* |
| function (% VC) | | | | |

#Hazard ratio and 95% confidence interval (95% CI) in multivariate Cox proportion hazard model analysis
*p < 0.05
LCL: Lower confidence limit
UCL: upper confidence limit

Cancer micro environment plays a great role in growth of cancer and malignant alteration of cancer cells. Especially, fibroblast and mesenchymal stem cell in stromal tissue of cancer, epithelial-mesenchymal transition (EMT) from cancer cells, or mesenchymal stem cell-derived activated cancer-associated fibroblast (CAF) are heavily involved in cancer progression and malignant alteration of cancer cells. Pirfenidone inhibits transforming growth factor-β (TGF-β), which activates the cancer micro environment, resulting in suppression of activated CAF and prevention of cancer under a pre-cancerous stage. This Pirfenidone's efficacies on prevention of cancer can be confirmed by known experimental methods.

### INDUSTRIAL APPLICABILITY

This invention provides the medicament for treatment of cancer chemoprevention which comprises pirfenidone as a pharmaceutical active ingredient. In another embodiment, there are provided the use of pirfenidone for the production of a medicament containing pirfenidone for cancer prevention, and methods for cancer chemoprevention comprising administering an effective amount of pirfenidone for cancer prevention to mammals including human.

1-11. (canceled)

12. A method of cancer chemoprevention which comprises a step of administering an effective amount of pirfenidone for cancer prevention to mammals including human.

13. A method of cancer chemoprevention which comprises a step of administering an effective amount of pirfenidone for cancer prevention to a subject who has a high risk factor of carcinogenesis.

14. The method of cancer chemoprevention according to claim 13, wherein said high risk factor of carcinogenesis is idiopathic pulmonary fibrosis.

15. The method of cancer chemoprevention according to claim 12, wherein any other pharmaceutical active drug is further administered as a concomitant drug.

16. The method of cancer chemoprevention according to claim 15, wherein said any other pharmaceutical active drug is a drug which prevents carcinogenesis, inhibits indirectly growth of cancer and/or directly inhibits growth of cancer.

17. The method of cancer chemoprevention according to claim 15, wherein said any other pharmaceutical active drug is used as adjuvant chemotherapeutic drug.

18. A method of cancer chemoprevention which comprises a step of administering an effective amount of 5-methyl-1-phenyl-2-(1H)-piridone for cancer prevention to mammals including human.

19. A method of cancer chemoprevention of lung cancer which comprises a step of administering an effective amount of pirfenidone for cancer prevention to mammals including human.
